(19) United States
(12) Patent Application Publication
(10) Pub. No.: US 2007/0154963 A1
(43) Pub. Date: Jul. 5, 2007

(54) Method to determine whether a compound is a cellular GSTpi inhibitor

(76) Inventors: Danying Cai, Redwood City, CA (US); Li Fang, Foster City, CA (US)

Correspondence Address: FOLEY & LARDNER LLP, 1530 PAGE MILL ROAD, PALO ALTO, CA, 94304, US

(21) Appl. No.: 11/323,810

(22) Filed: Dec. 30, 2005

(51) Int. Cl. G01N 33/574

(52) U.S. Cl. 435/7.23

(57) ABSTRACT

A method to determine whether a compound is a cellular GSTπ inhibitor.

### BACKGROUND OF THE INVENTION

1. Field of the Invention

This invention relates to a method to determine whether a compound is a cellular glutathione S-transferase (GST) π inhibitor.

2. Description of the Related Art

U.S. Pat. Nos. 5,599,903; 5,763,570; 5,767,086; 5,786,336; and 5,955,432; European Patent Publication No. 0 645 397; and PCT International Publications Nos. WO 95/08563 and WO 96/40205 disclose various tripeptide and tetrapeptide compounds that are analogs of reduced glutathione (L-γ-glutamyl-L-cysteinylglycine), including compounds of the formula [WO 95/08563]: 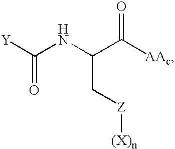
and their C₁₋₁₀ alkyl or alkenyl or C₇₋₁₂ aralkyl esters, amides, and mixed ester/amides, where:
Z is S, O, or C
n is 1 to 3;
when Z is S or O and n is 1, X is a C₁₋₂₀ hydrocarbyl optionally containing 1 or 2 non-adjacent O, S, or N heteroatoms, unsubstituted or mono- or disubstituted with halo, —NO, —NO₂, —NR₂, —OR, or —SR,
where R is H or C₁₋₄ alkyl;
when Z is S and n is 2, one X is as above defined and the other X is C₁₋₄ alkyl; and
when Z is C and n is 3, one X is as above defined and the other two X are independently H or C₁₋₄ alkyl;
YCO is γ-glu, β-asp, glu, asp, γ-glu-gly, β-asp-gly, glu-gly, asp-gly; and
AA_{c} is an amino acid coupled through a peptide bond to the remainder of the compound.

The compounds are described as having various uses, including as reagents useful in characterizing GST isoenzymes, in determining the GST complements of cells and tissues, as chromatographic affinity ligands, binding agents, and enzyme inhibitors; and therapeutically to: potentiate the cytotoxic effects of chemotherapeutic agents in tumor cells, selectively exert cytotoxicity in tumor cells, elevate the production of granulocyte-macrophage progenitors in bone marrow, stimulate the differentiation of bone marrow cells, mitigate the bone marrow-destructive effects of chemotherapeutic agents, and modulate hematopoiesis in bone marrow.

TLK117, identified in those patents and publications as TER 117 and named variously as γ-Glu-Cys(Bz)-phenylGly, γE-C(Bz)-φG, γE-C(Bz)-PG, γE-C(benzyl)-φG, and benzyl PG, is one of these compounds. TLK117 is the compound of the formula 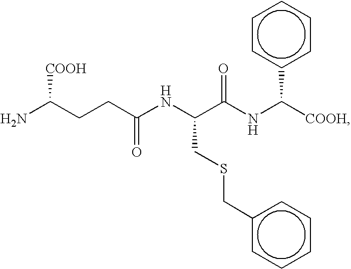
and may be named L-γ-glutamyl-S-(phenylmethyl)-L-cysteinyl-D-phenylglycine. TLK199, identified in those patents and publications as TER 199, is the diethyl ester of TLK117.

The GST P1-1 inhibitory activity of glutathione analogs such as TLK117 is usually measured in an enzymatic assay using purified GST P1-1 isoenzyme; and in such an assay TLK117 inhibits GST P1-1 with an IC₅₀ of approximately 400 nM. TLK199 is inactive (IC₅₀>200 μM) in this assay.

However, diacids such as TLK117 are known to have poor permeability through cell membrane and hence show poor activity in whole cell assays, while the cell membrane permeability is much higher with diesters such as the diethyl esters (Hamilton et al., J. Biol. Chem., 267, 24933-24936 (1992); Levy et al., Proc. Nat'l Acad. Sci. U.S.A., 90, 9171-9175 (1993); Sharkey et al., Cancer Chemother. Pharmacol., 46, 156-166 (2000)). Therefore, diesters such as the diethyl esters are used when these glutathione analogs are investigated for their effects on cells or are considered as potential chemotherapeutic agents. These diesters are hydrolyzed to diacids within the cell.

GSTπ overexpression has been observed in a number of tumor cell lines and has been correlated with resistance to chemotherapy (Oakley et al., J. Mol. Biol., 274, 84-100 (1994)).

Many conditions are characterized by depleted bone marrow, including myelodysplastic syndrome (MDS), a form of pre-leukemia in which the bone marrow produces insufficient levels of one or more of the three major blood elements (white blood cells, red blood cells and platelets). A reduction in blood cell levels and the generation of new blood cells in the bone marrow, myelosuppression, is also a common, toxic effect of many standard chemotherapeutic drugs.

TLK199 has been shown to induce the differentiation of HL-60 promyelocytic leukemia cells in vitro, to potentiate the activity of cytotoxic agents both in vitro and in vivo, and to stimulate colony formation of all three lineages of hematopoietic progenitor cells in normal human peripheral blood. In preclinical testing, TLK199 has been shown to increase white blood cell production in normal animals as well as in animals in which white blood cells were depleted by treatment with cisplatin or fluorouracil. Similar effects may provide a new approach to treating MDS. TLK199 is currently being evaluated in a clinical trial for the treatment of MDS. Interim results from this trial, reported at the 2004 and 2005 American Society of Hematology meetings, demonstrated that TLK199 was well tolerated and resulted in multilineage hematologic improvement. These results also suggest a potential role for TLK199 in treating chemotherapy-induced cytopenias.

Because of the desirability of finding new GSTπ inhibitors for the treatment of the diseases mentioned above, it would be desirable to develop a convenient method to determine whether a compound is a cellular GSTπ inhibitor.

The disclosures of the documents referred to in this application are incorporated into this application by reference.

### SUMMARY OF THE INVENTION

This invention is a method to determine whether a compound is a cellular GSTπ inhibitor, comprising:
(a) contacting a cell with the compound;
(b) lysing the cell; and
(c) measuring the amount of immunoprecipitable GSTπ in the cell lysate;
   where an amount of immunoprecipitable GSTπ in the cell lysate that is lower than the amount of immunoprecipitable GSTπ in the lysate of a comparable cell that has not been contacted with the compound shows that the compound is a cellular GSTπ inhibitor.

This invention is advantageous in offering a direct method of determining whether a compound is a cellular GSTπ inhibitor, and is a convenient “bridge” between enzymatic assays (which, in the case of glutathione analogs, are responsive primarily to the diacids) and animal assays (which, in the case of glutathione analogs, are responsive primarily to the diesters due to the limited ability of the diacids to cross cell membranes). The cellular GSTπ inhibitors identified by the method of this invention can be used for the uses disclosed for TLK199 in the “Background to the Invention” section of this application.

Preferred embodiments of this invention are characterized by the specification and by the features of claims 2-11 of this application as filed.

### BRIEF DESCRIPTION OF THE DRAWINGS

FIG. 1 is a scanned image of a Western blot showing cellular GSTπ inhibition in an HL-60 cell by TLK199 at various concentrations.

FIG. 2 is a graph showing the dependence of cellular GSTπ inhibition on TLK199 concentration, using band intensities from the Western blot of FIG. 1.

### DETAILED DESCRIPTION OF THE INVENTION

Definitions

“Immunoprecipitable GSTπ” means that part of the GSTπ content of a cell that, in lysate from that cell, is capable of binding to an antibody selective for GSTπ. “Cellular GSTπ inhibition” means the reduction in the amount of immunoprecipitable GSTπ within a cell by contacting that cell with a compound; and a “cellular GSTπ inhibitor” is a compound that causes cellular GSTπ inhibition.

The method of this invention.

The method of this invention comprises contacting a cell with a compound to be tested, and subsequently lysing the cell and measuring the amount of immunoprecipitable GSTπ in the cell lysate.

The cell used in the method is a cell that expresses GSTπ at a detectable level. Desirably the expression is at a high level to increase the sensitivity of the method; but the starting cell number for the method can be adjusted according to the level of GSTπ expression in the cell (increased for lower expressing cells) provided that an adequate signal level can be obtained for visualization/quantitation. A high level of GSTπ expression is characteristic of many human tumor cells, such as the HL-60 human promyelocytic leukemia cell and the DLD-1 human colon adenocarcinoma cell; and cells such as these are particularly suitable for the method of this invention. The cell culture medium used should be suitable for supporting growth of the cell used.

The cell will normally be contacted with the compound for a time between 10 minutes and 100 hours, for example between 0.5 and 5 hours, such as 2 hours. A longer contacting time will increase the sensitivity of the method, while a shorter time may be more convenient and may also be more representative of the contacting time that might be experienced in vivo if the compound were to be used as a therapeutic agent. The compound will normally be used in solution in any suitable solvent; for example in aqueous solutions such as water or phosphate-buffered saline, or in organic solutions such as lower alcohols, e.g. ethanol, or dimethyl sulfoxide (as in the example).

The compound to be tested for cellular GSTπ inhibition may be tested at a single concentration, for example at a concentration selected by comparable testing with one or more known cellular GSTπ inhibitors such that the known inhibitor(s) provide quantitatable inhibition at that concentration. For example, compounds could be tested at 20 μM, since the known cellular GSTπ inhibitor TLK199 shows approximately 90% inhibition of cellular GSTπ at that concentration. In this manner, compounds can be rapidly screened to determine whether they possess at least minimal cellular GSTπ inhibitory activity. Alternatively or additionally the compound may be tested at a range of concentrations, and the concentration dependence of the cellular GSTπ inhibition measured so that, for example, the IC₅₀, the concentration that causes 50% inhibition of cellular GSTπ, can be determined, and the cellular GSTπ inhibitory potency of different compounds can be compared.

If an adherent cell is used in the method, the compound contacting step may be performed in the same manner as for a non-adherent cell. Following the compound treatment, the cell may then be washed while still adherent to the surface, and then be detached from the surface by conventional methods such as incubating with trypsin for a few minutes. The remaining steps of the process may then be followed just as for a non-adherent cell (lysis, immunoprecipitation, and visualization).

The anti-human GSTπ antibody used for immunoprecipitation in the example here is a purified monoclonal mouse anti-human GSTπ antibody, clone 353-10, from DakoCytomation. Similar anti-human GSTπ antibodies are available from other vendors such as Diagnostic Biosystems, Pleasanton, Calif., U.S.A. and Abcam plc, Cambridge, England. The anti-human GSTπ antibody used for the detection of GSTπ in the Western blot or dot-blot analysis (the primary detection antibody) may be any anti-human GSTπ antibody suitable for Western blot or dot-blot analysis provided that it is raised in a species different from the species in which the immunoprecipitation antibody is raised. This primary detection antibody may be conjugated to a label if desired, avoiding the need for a secondary detection antibody (a visualization antibody), but more usually it will be unlabeled. The secondary detection antibody may be any antibody raised against the species of the primary detection antibody, and will be conjugated to some detectable label to enable visualization/quantitation of the immunoprecipitated GSTπ. Suitable labels are fluorescent dyes (e.g. infrared fluorescent dyes such as the IRDye™ 800 used in the visualization antibody of the example), and enzymes such as horseradish peroxidase or alkaline phosphatase (detectable by color reagents or chemiluminescence).

If desired, the proteins from the cell lysate may be separated by methods such as sodium dodecyl sulfate-polyacylamide gel electrophoresis (SDS-PAGE) and transferred to a membrane (Western blot), as shown in the example. However, because the assay method has a relatively low background, the method can be performed without separating the proteins by directly spotting them onto a membrane (“dot-blot”) for visualization/quantitation. The electrophoresis and Western blot or dot-blot analysis are carried out by techniques well-known to a person of ordinary skill in the art that are described in references such as “Handbook of Bioseparations”, Ahuja, ed., Academic Press, San Diego, Calif., 2000. If the proteins are separated, each of the two immunoprecipitated GST P1-1 dimer forms, the inter(polypeptide chain) disulfide-bonded dimer and the non-disulfide-bonded dimer (which appears as GST P1 monomer on SDS-PAGE due to denaturation), may be separately visualized and quantitated, or the total immunoprecipitated GSTπ my be quantitated by summing the two sets of bands, as was done in the example. Preferably, either total imnmunoprecipitated GSTπ or immunoprecipitated GST P1-1 dimer are used when determining the amount of immunoprecipitated GSTπ. If the proteins are not separated, total immunoprecipitated GSTπ is used.

The following example is given to enable a person of ordinary skill in the art to more clearly understand and practice this invention. It should not be considered as limiting the scope of the invention, but merely as illustrating and representing the invention.

The human promyelocytic leukemia cell line HL-60 (Catalog # CCL-240) was obtained from the American Type Culture Collection, Manassas, Vir., U.S.A. TLK199 was obtained from Telik, Inc., and was prepared as a solution at 40 mM in dimethyl sulfoxide DMSO). This stock solution was diluted with DMSO to give further solutions of 20 mM, 10 mM, 5 mM, and 1 mM TLK199 in DMSO. RPMI 1640 medium, NuPAGE® LDS sample buffer (non-reducing lithium dodecyl sulfate buffer, Catalog # NP0007), and NuPAGE® Novex 4-12% Bis-Tris polyacrylamide gels (Catalog # NP0321) were obtained from Invitrogen, Inc., Carlsbad, Calif., U.S.A. Complete Mini protease inhibitor cocktail tablets (Catalog # 04 693 124 001) were obtained from Roche Diagnostics, Inc., Indianapolis, Ind., U.S.A., and were used according to package directions. Lysis buffer (50 mM Tris, pH 8.0, 120 mM NaCl, 0.5% NP-40, and 100 mM NaF) was prepared at Telik, Inc., and the protease inhibitors added shortly before use. The Bio-Rad protein assay kit (Catalog # 500-0001) was obtained from Bio-Rad Laboratories, Hercules, Calif., U.S.A. Purified monoclonal mouse anti-human GSTπ antibody, clone 353-10 (Catalog # M0529) was obtained from DakoCytomation, Inc., Carpinteria, Calif., U.S.A. Immunopure® immobilized Protein A/G agarose beads (Catalog # 20421) were obtained from Pierce, Rockford, Ill., U.S.A. Blocking buffer (Catalog # 927-40003 or 927-40010) and the Odyssey™ infrared fluorescence scanner were obtained from Li-Cor Biosciences, Lincoln, Nebr., U.S.A. Rabbit polyclonal anti-human GSTπ antibody (Catalog # 354212) was obtained from EMD Biosciences, Inc., La Jolla, Calif., U.S.A. IRDye™ 800CW conjugated affinity purified goat anti-rabbit IgG (H&L) (Catalog # 611-132-122) was obtained from Rockland Inmmunochemicals, Inc., Gilbertsville, Pa., U.S.A.

HL-60 cells were seeded at 6×10⁵ cells/mL in 20 mL RPMI 1640 medium supplemented with 10% fetal bovine serum, 1 mM L-glutamine and 20 μM gentamycin in each of six T75 flasks, and incubated at 37° C. overnight in a 5% CO₂ atmosphere. To each flask was then added 20 μL of DMSO, or 20 μL of one of the TLK199 DMSO solutions described above, to give final TLK199 concentrations of 0, 1, 5, 10, 20, and 40 μM; and the cells incubated for a further two hours. The contents of each flask were then treated as follows: the cells were pelleted by centrifugation and the cell pellet washed twice with phosphate-buffered saline. The cells were then lysed by suspending the pellet in 1 mL lysis buffer, and incubating with gentle agitation at 4° C. for 30 minutes. The cell lysate was cleared by centrifugation at 13,000 rpm for 10 minutes, and the protein content of the lysate was measured using the Bio-Rad protein assay kit. Cell lysate containing 1 mg total proteins was incubated at 4° C. overnight with 4.2 μg of purified mouse monoclonal anti-human GSTπ antibody in 1 mL total volume. Protein A/G agarose beads, 50 μL, were added to the mixture and incubated at 4° C. for 1 hour. The beads were collected by centrifugation, washed three times in lysis buffer, and resuspended in 10 μL 1× non-reducing lithium dodecyl sulfate buffer, and the suspension heated at 95° C. for 5 minutes. The proteins from the six cell lysates, together with a molecular weight marker, were then separated by electrophoresis on a 4-12% Bis-Tris gel and transferred to a nitrocellulose membrane. Standard Western blot procedures were followed in the subsequent steps. The membrane was incubated first with blocking buffer at room temperature for 1 hour, then with rabbit polyclonal anti-human GSTπ antibody (1:2000 dilution), and finally with IRDye™ 800-conjugated, affinity purified goat anti-rabbit IgG (H+L) (1:10,000 dilution). The GSTπ protein bands were visualized and the summed intensities of the GST P1-1 dimer and GST P1 monomer bands quantitated on an Odyssey infrared scanner.

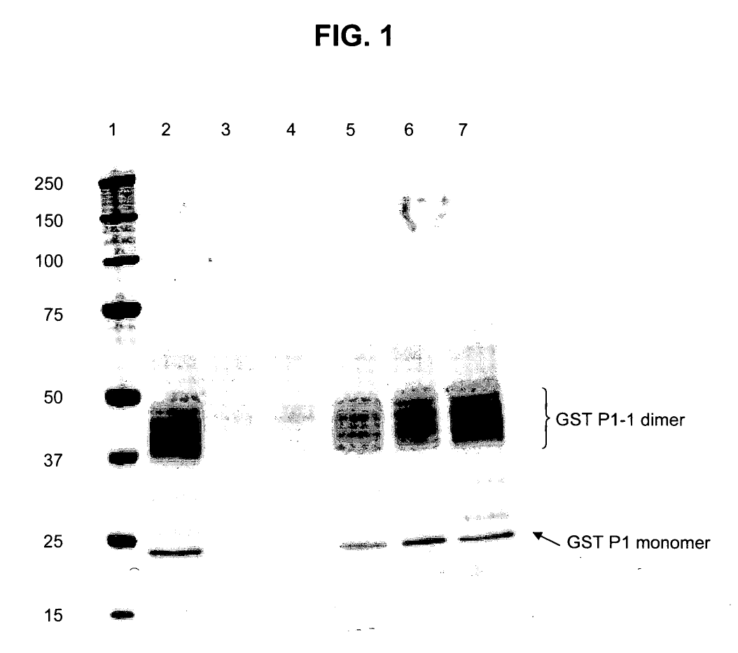

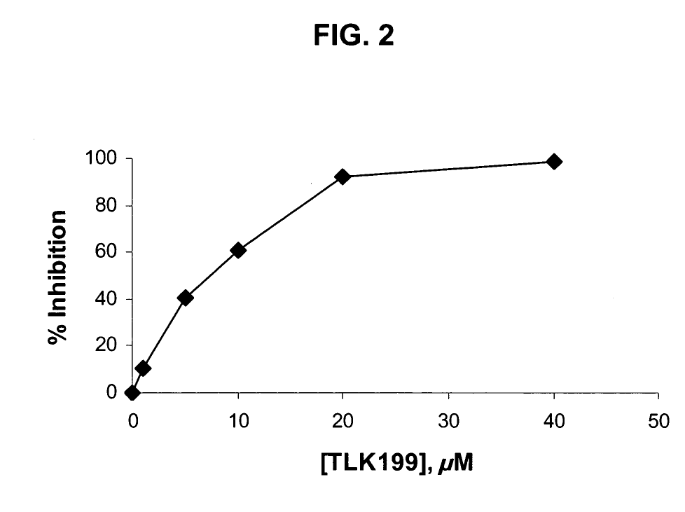

The resulting data are shown in FIGS. 1 and 2.

FIG. 1 is a scanned image of the Western blot, showing cellular GSTπ inhibition by TLK199. Lane 1 is a molecular weight marker, with the molecular weights in kiloDaltons of the markers given on its left; lane 2 is the lysate from cells contacted only with DMSO (the control lane); lane 3 is the lysate from cells contacted with 40 μM TLK199; lane 4 is the lysate from cells contacted with 20 μM TLK199; lane 5 is the lysate from cells contacted with 10 μM TLK199; lane 6 is the lysate from cells contacted with 5 μM TLK199; and lane 7 is the lysate from cells contacted with 1 μM TLK199. The bands containing GST P1-1 dimer and GST P1 monomer are indicated on the right.

FIG. 2 is a graph showing the dependence of cellular GSTπ inhibition on TLK199 concentration, using the summed band intensities from the Western blot of FIG. 1. The percentage inhibition for a particular sample was calculated from the equation:
% inhibition=100×[1−(intensity of sample band)/(intensity of DMSO control band)].

The figures show that TLK199 is a cellular GSTπ inhibitor, because the band intensities for (i.e. the amounts of immunoprecipitable) both GST P1 and GST P1-1 are lower in the cells contacted with TLK199 than in cells contacted only with DMSO, and show also that the cellular GSTπ inhibitory effect of TLK199 is concentration dependent.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1-11. (canceled)

12. A method to determine whether a compound is a cellular GSTπ inhibitor, comprising:
(a) contacting a cell with the compound;
(b) lysing the cell; and
(c) measuring the amount of immunoprecipitable GSTπ in the cell lysate;
where an amount of immunoprecipitable GSTπ in the cell lysate that is lower than the amount of immunoprecipitable GSTπ in the lysate of a comparable cell that has not been contacted with the compound shows that the compound is a cellular GSTπ inhibitor.

13. The method of claim 12 where the GSTπ is total GSTπ.

14. The method of claim 12 where the GSTπ is GST P1-1.

15. The method of claim 12 where the cell is a GSTπ-expressing human tumor cell.

16. The method of claim 15 where the cell is an HL-60 cell.

17. The method of claim 12 where the cell is contacted with the compound for a time between 10 minutes and 100 hours.

18. The method of claim 17 where the cell is contacted with the compound for a time between 0.5 and 5 hours.

19. The method of claim 18 where the cell is contacted with the compound for a time of 2 hours.

20. The method of claim 12 where proteins in the cell lysate are separated by SDS-PAGE and the immunoprecipitable GSTπ is detected by Western blot.

21. The method of claim 20 where the immunoprecipitable GSTπ is measured by infrared fluorescence.

22. The method of claim 12 where the percentage inhibition of cellular GSTπ by the compound is determined.

23. The method of claim 22 where the percentage inhibition is determined at multiple concentrations of the compound.

24. The method of claim 23 where the IC₅₀ for inhibition of cellular GSTπ by the compound is determined.
